(19) United States
(12) Patent Application Publication
(10) Pub. No.: US 2025/0152404 A1
(43) Pub. Date: May 15, 2025

(54) OSTOMY IMPLANT DEVICE AND METHOD

(71) Applicants: BOSTON SCIENTIFIC MEDICAL DEVICE LIMITED, Galway (IE); BOSTON SCIENTIFIC SCIMED, INC., Maple Grove, MN (US)

(72) Inventors: Aditya DHANOTIYA, Indore (IN); Arun ADHIKARATH BALAN, Aluva (IN); Pooja Bhuvanesh KULKARNI, Pune (IN); Nitesh Ghananil BAVISKAR, Kalyan West (IN); Deepak Kumar SHARMA, Muzaffarnagar (IN); Sharath Kumar G, Kanakapura (IN); Hitendra PUROHIT, Vadodara (IN); Marco Vinicio ARIAS ALPIZAR, San José (CR); Gabriela GAMBOA BASTOS, San José (CR); Fernando VALERIO GUERRERO, Alajuela (CR); Carolina PADILLA CASTRO, San José (CR); Mauro VÍQUEZ GONZALEZ, Heredia (CR); Valeria ZÚÑIGA VARGAS, San Francisco (CR)

(21) Appl. No.: 18/930,573

(22) Filed: Oct. 29, 2024

Related U.S. Application Data
(60) Provisional application No. 63/599,406, filed on Nov. 15, 2023.

(51) Int. Cl. A61F 5/448; A61F 2/90; A61F 5/44

(52) U.S. Cl. CPC A61F 5/448; A61F 2/90; A61F 5/4408

(57) ABSTRACT

An ostomy device includes an implant including a body sized and shaped for implantation in a stoma. The body includes a first portion defining a channel extending from a first end to a second end. The first end is configured to interface with a lumen interior to an abdomen. The second end is configured to provide a path from the lumen through the channel to an exterior of the abdomen. The implant further includes a flange attached to an exterior of the body. The flange includes a collar extending from a first rim attached to an exterior of the body to a second rim attachable to a skin adjacent to the stoma.

### PRIORITY CLAIM

The present disclosure claims priority to U.S. Provisional Patent Application Ser. No. 63/599,406 filed Nov. 15, 2023; the disclosure of which is incorporated herewith by reference.

### FIELD

The present disclosure relates to ostomy devices comprising an implant for post-procedural ostomy management.

### BACKGROUND

An ostomy generally refers to a surgical procedure in which an opening, or stoma, is created in the abdomen to provide a path for human waste or a stool (e.g., a liquid waste, a solid waste, and/or a gas waste) to be removed from the body. Sections of the gastrointestinal tract are bypassed, and the waste typically produced and transported through the body is excreted through the stoma. Traditionally, ostomies are managed through technology such as an ostomy bag, which the patient must regularly vacate and replace. Ostomy patients may experience complications with the stoma including skin irritation, stoma leaks, bleeding, and other issues.

### SUMMARY

The present disclosure relates an ostomy device which includes an implant. The implant includes a body sized and shaped for implantation in a stoma. The body includes a first portion defining a channel extending from a first end to a second end. The first end is configured to interface with a lumen interior to an abdomen. The second end is configured to provide a path from the lumen through the channel to an exterior of the abdomen. The implant further includes a flange attached to an exterior of the body. The flange includes a collar extending from a first rim attached to an exterior of the body to a second rim attachable to a skin adjacent to the stoma.

In an embodiment, the body comprises a living hinge between the first portion and a second portion and wherein the living hinge is biased to bend so that the second portion assumes a flared shape.

In an embodiment, the device further includes a sheath around the body configured to restrict the living hinge from bending during insertion of the implant.

In an embodiment, the sheath is removed after the implant is inserted so that the living hinge bends and the second portion flares to contact intestinal walls adjacent to the lumen.

In an embodiment, the device further includes a rubber ring disposed between the second rim and the skin to provide a lining for leakage prevention.

In an embodiment, the device further a stoma plug configured to insert in the channel at the second end of the body.

In an embodiment, the stoma plug comprises a plug body and a membrane, the membrane including slits to enable gas to pass from the lumen through the channel.

In an embodiment, the stoma plug is press fit into the channel and removable by proving a radial force on the body to deform the body and push the stoma plug out of the channel.

In an embodiment, an ostomy bag is attached around the second end of the body and the stoma plug is pushed into the ostomy bag.

In an embodiment, the stoma plug is screwed, snap fit or clasped to the body.

In an embodiment, the first rim is screwed or glued to the body and the second rim is sutured to the skin adjacent to the stoma.

In addition, the present disclosure relates to an ostomy device which includes a stent. The stent is sized and shaped for implantation in a hole in an abdomen. The stent comprises a tubular body defining a channel extending from a first end to a second end. The first end is configured to interface with a lumen interior to the abdomen. The second end is configured to provide a path from the lumen through the channel to an exterior of the abdomen. The stent is configured to be attached to the hole in the abdomen.

In an embodiment, the stent comprises a first flange configured to contact an inner surface of the abdomen and a second flange configured to contact an outer surface of the abdomen.

In an embodiment, the first end of the stent is stitched to the lumen.

In an embodiment, the second end includes a coupling mechanism configured to be attached to an ostomy bag.

In addition, the present disclosure relates to a method. The method includes implanting an implant of an ostomy device in a stoma, the implant comprising a body including a first portion defining a channel extending from a first end to a second end, the first end configured to interface with a lumen interior to an abdomen, the second end configured to provide a path from the lumen through the channel to an exterior of the abdomen, wherein the implant further includes a flange attached to an exterior of the body, the flange including a collar extending from a first rim attached to an exterior of the body to a second rim; and attaching the second rim to a skin adjacent to the stoma.

In an embodiment, the body comprises a living hinge between the first portion and a second portion and wherein the living hinge is biased to bend so that the second portion assumes a flared shape.

In an embodiment, the ostomy device further includes a sheath around the body configured to restrict the living hinge from bending during insertion of the implant.

In an embodiment, the method further includes removing the sheath after the implant is inserted so that the living hinge bends and the second portion flares to contact intestinal walls adjacent to the lumen.

In an embodiment, the ostomy device further includes a rubber ring disposed between the second rim and the skin to provide a lining for leakage prevention.

### BRIEF DESCRIPTION

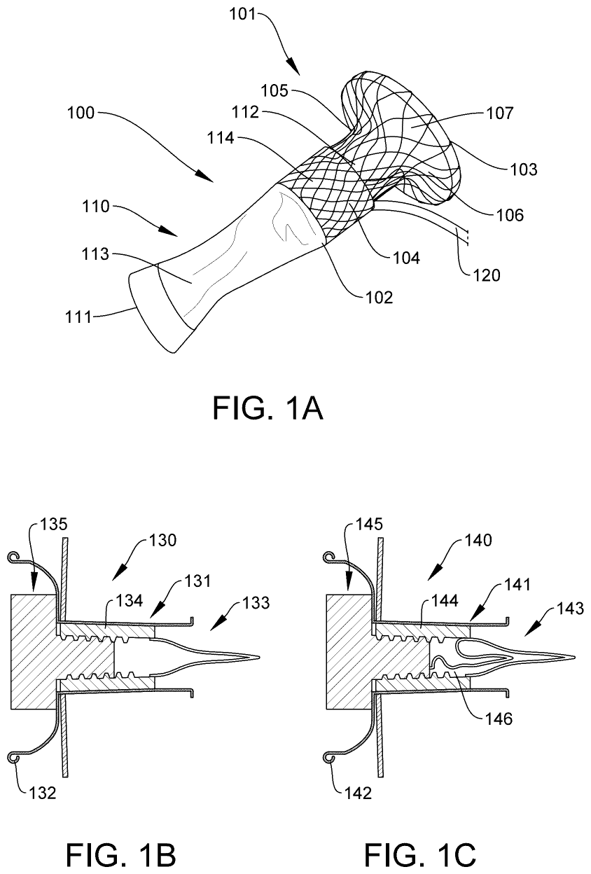

FIG. 1a shows an ostomy device including a stent for implantation within a stoma and a balloon attached to the stent that can be transitioned between a closed state to prevent waste from being expelled through the stoma and an open state to allow the evacuation of the waste.

FIG. 1b shows an ostomy device including a cap according to various exemplary embodiments.

FIG. 1c shows an ostomy device including a cap with an inflation tube extending proximally from the cap to a balloon according to various exemplary embodiments.

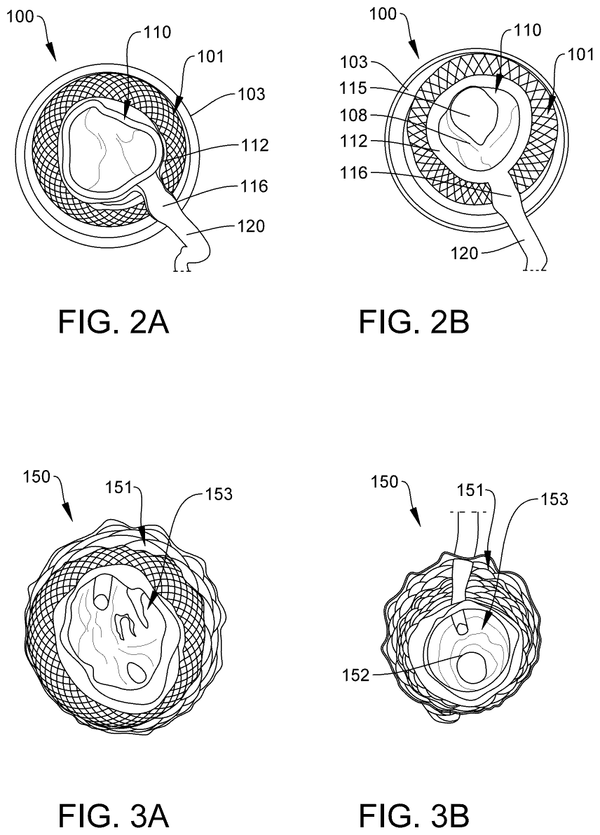

FIG. 2a shows a side view of the device of FIG. 1 with the balloon deflated and the channel through the stent closed.

FIG. 2b shows a side view of the device of FIG. 1 with the balloon inflated and the channel through the stent opened.

FIG. 3a shows a side view of an ostomy device including a stent placed external to a lumen of the intestine with the balloon deflated and the channel through the stent closed.

FIG. 3b shows a side view of the device of FIG. 3a placed external to the lumen with the balloon inflated and the channel through the stent opened.

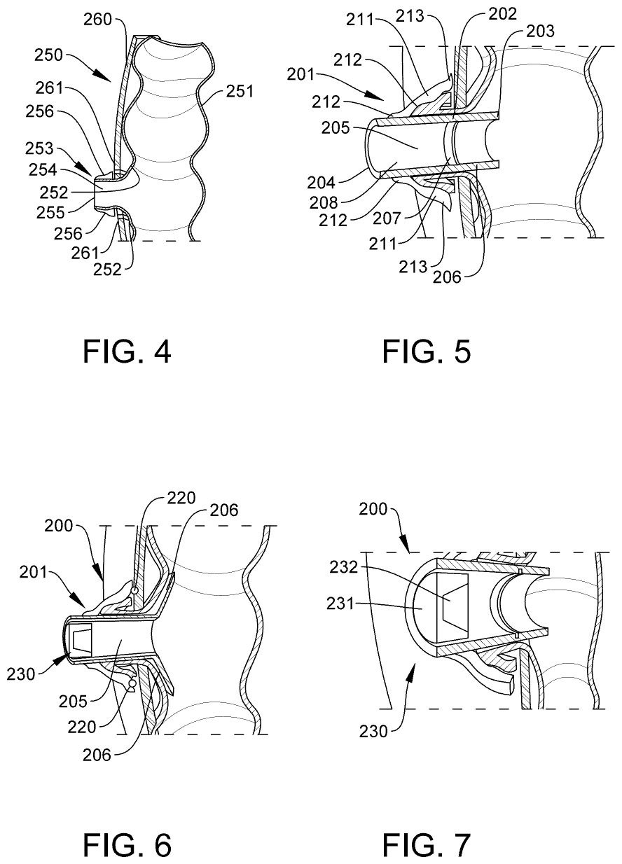

FIG. 4 shows an anatomy of a patient including the intestines, a stoma, and skin of the abdominal wall.

FIG. 5 shows an implant of the device including a body defining a channel and an external flange for attaching the implant to the patient anatomy.

FIG. 6 shows the device including the implant of FIG. 5 with the body in the deployment state, e.g., with the living hinge bent so that the first portion (proximal portion) of the body can expand into a flared shape.

FIG. 7 shows a stoma plug of the device of FIG. 6.

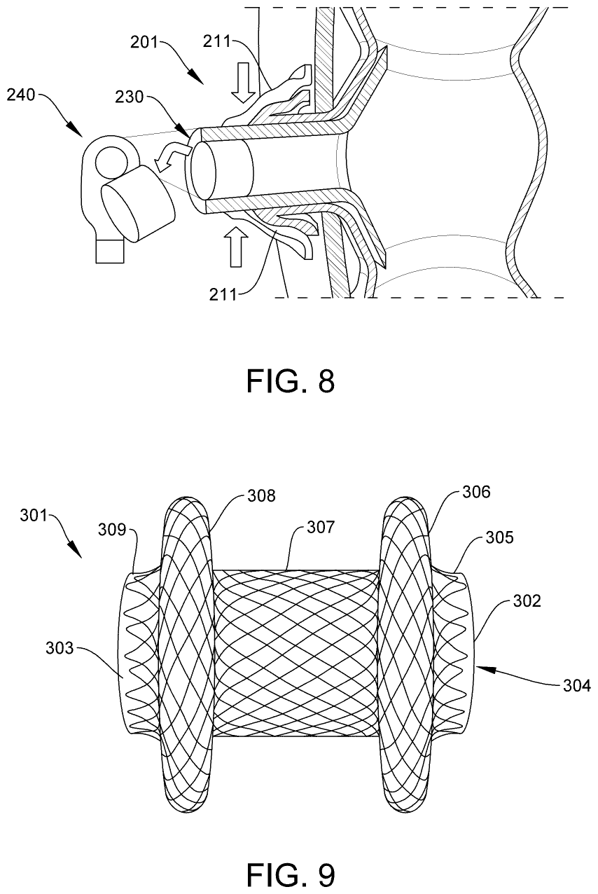

FIG. 8 shows the removal of the stoma plug of FIG. 6 for the passing of waste.

FIG. 9 shows a stent of the device defining a channel from a first end (e.g., proximal end) for connecting to the intestines to a second end (e.g., distal end) for connecting to the environment external to a hole in a patient's abdomen.

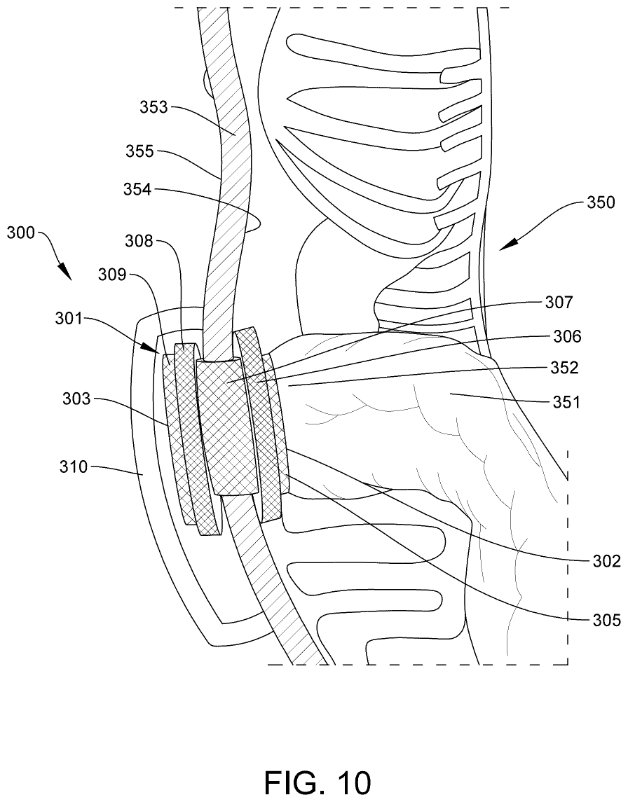

FIG. 10 shows the device including the stent connected to a patient anatomy.

### DETAILED DESCRIPTION

The present disclosure may be further understood with reference to the following description and the appended drawings, wherein like elements are referred to with the same reference numerals. The present disclosure relates to ostomy devices comprising an implant for post-procedural ostomy management. In some embodiments, the exemplary ostomy device can be attached to a stoma to provide a channel through which waste is passed. The channel may be selectively blocked to prevent the flow of waste and may be opened by a user at a desired time to expel the waste.

In other embodiments, the exemplary ostomy device is attached directly to the intestine at one end and to an opening in the abdomen at the other end to provide the channel through which waste is passed. A first exemplary embodiment of an ostomy device (along with alternatives thereto) is described with regard to FIGS. 1a-c, 2a-b, and 3a-b; a second exemplary embodiment of an ostomy device and its alternatives is described with regard to FIGS. 4-8; and a third exemplary embodiment of an ostomy device and its alternatives is described with regard to FIGS. 9-10. It should be understood that various principles described herein with regard to various features of the exemplary ostomy devices can be incorporated into other types of ostomy devices and the exemplary embodiments are not limited to the specific configurations shown in the Figures, as will be described in greater detail below and that the various features of the embodiments may be combined in any manner not inconsistent with the operation of the various devices.

It is noted that the terms proximal and distal as used herein refer to the anatomy of the patient wherein “proximal” refers to a direction toward the interior of the body and “distal” refers to a direction toward the exterior of the body. With regard to the exemplary ostomy devices described herein, a proximal side of the device generally refers to the side that interfaces with the intestine and a distal side of the device generally refers to the side that interfaces with the environment exterior to the patient.

There are several types of ostomy procedures including a colostomy, an ileostomy and a urostomy. In a colostomy, portions of the large intestine (e.g., colon and/or rectum) are removed and the remaining tissue is brought through an opening in the abdomen to form the stoma. In an ileostomy, the ileum (end of the small intestine) is brought through the abdomen to form the stoma. After any of these procedures is performed, waste can be passed through the stoma, e.g., into an ostomy bag.

In a urostomy, a portion of the small intestine is detached from the rest of the small intestine and brought through the abdomen to form a stoma. This detached portion of the small intestine is coupled to the ureters to allow for the passage of urine away from the bladder and out of the stoma to an ostomy bag. Some ostomy procedures can be performed laparoscopically using small incisions while others are performed as open surgeries using a larger incision. Some ostomy procedures are permanent, e.g., in the case of bowel cancer or serious injury, while others are reversible, e.g., while the bowel recovers from events such as infection, inflammation or stab wounds. The stoma may be flat against the skin or protrude a small distance out the abdomen.

Typically, post-procedural ostomy management includes the use of an ostomy bag. Such bags are generally attached to the stoma to collect waste which may then be evacuated at appropriate times. Different procedures may require different durations for which the ostomy bag must be used and, as mentioned above, some ostomies are permanent. Particularly for patients with permanent stomas, the wearing of an ostomy bag may have serious psychosocial implications and can lead to feelings of stigma, fear, questioning and isolation, all of which may significantly impact the patient's quality of life.

Ostomy patients may experience complications with the stoma including: skin irritation; stoma leaks; bleeding; retracted or prolapsed stoma; parastomal hernia; blockages or bowel obstruction; and/or necrosis. When an ostomy bag is used, the ostomy patient may experience problems with the bag including: gas leakage (and the associated odor); leakage of waste at the junction between the stoma, skin, and the bag opening; a weight sensation when the bag is filled; an undesirable bag shape that is not adjusted for different body types (convex-concave); multiple steps for cleaning, attaching the adhesive layer, emptying the bag, cleaning the bag, etc.; visual unattractiveness, particularly when the bag fills with gas (can look like a balloon under the clothes); the bag getting wet during a shower; and/or skin infection due to the use of adhesive. Accordingly, the existing procedures for post-operative stoma management leave room for improvement.

According to various exemplary embodiments described herein, an implant can be attached to the stoma, tissue surrounding the stoma, or a hole in the abdomen (e.g., when a stoma is not created) to provide a channel from the intestine to the environment exterior to the patient for evacuation of waste. Various ostomy devices are described herein. In some embodiments, the channel through the implant may be closed so that the patient does not regularly need to wear an ostomy bag and open the channel through the implant at a desired time to evacuate waste that has accumulated in the intestine. In some embodiments, an ostomy bag can be attached to the implant as needed at the desired time and removed when the channel through the implant is closed.

An ostomy device according to one embodiment includes an implant with a balloon that can be inflated to open a channel through the implant for waste evacuation or deflated to close the channel and prevent waste from passing through the implant. In one embodiment, the implant comprises a stent-like device (referred to herein as a “stent” for ease of description) comprising a wire mesh that forms a tubular body extending from a first end (e.g., proximal end) to a transition region and forms a flared region extending from the transition region to a second end (e.g., distal end). The wire mesh is covered to define a closed channel through the stent through which waste can travel. The flared region can be stitched to the stoma and/or skin surrounding the stoma and the tubular body can extend into the lumen of the intestine. The balloon can be attached to the stent around the body and extend proximally off the first end. The balloon can, for example, be substantially tubular with two open ends when inflated. A first portion of the balloon of such embodiments extends proximally off the first end of the stent while a second portion is wrapped around the body of the stent so that, when the balloon is deflated, the first portion collapses proximal to the stent to block the channel through the stent while the second end remains wrapped around the body, as will be described in greater detail below.

FIG. 1a shows an ostomy device 100 including a stent 101 for implantation within a stoma and a balloon 110 attached to the stent 101 that can be transitioned between a closed state to prevent waste from being expelled through the stoma and an open state to allow the evacuation of the waste. The stent 101 is sized and shaped for implantation within the stoma and for interfacing with the lumen of the intestine. In particular, the stent 101 extends from an open first end 102 (e.g., proximal end) that, when the device 100 is implanted in the patient, is located within the lumen of the intestine to an open second end 103 (e.g., distal end) that, when the device 100 is implanted in the patient, is located exterior to the stoma. The stent 101 comprises a mesh or stitching that forms a tubular body 104 extending the first end 102 to a transition region 105 and an enclosed flared portion 106 extending from a transition region 105 to the second end 103.

In this example, an interior of the stent 101 is covered via a cover 107 that defines an enclosed channel 108 (not labeled in FIG. 1 and shown in greater detail in FIGS. 2a-b) that extends through the body 104 of the stent 101 from the first end 102 to the transition region 105 (e.g., a volume through which stool may travel from the intestine through the body 104) and the flared portion 106 through which the waste may move distally out of the second end 103. The device 100 further includes a cap (not shown in FIG. 1) that can be selectively attached to and removed from the second end 103 to seal or open the distal opening and, in some embodiments, configured to attach of evacuation accessories to the device 100 as will be described in greater detail below.

The device 100 further includes a balloon 110 attached to the body 104 of the stent 101. When inflated, the balloon 110 of this embodiment is tubular and defines an interior channel 115 (not labeled in FIG. 1 and shown in greater detail in FIG. 2b) and two open ends permitting accessing the interior channel 115. In other words, when inflated, the balloon 110 is shaped so that a first cylindrical portion of the balloon 110 defines an “inner diameter” of the tube along its length and a second cylindrical portion of the balloon 110 outside the first circular portion defines an “outer diameter” of the tube along its length, with an inflation fluid (e.g., air) contained between the two portions defining a “wall thickness.”

In other words, the first and second portions of the balloon 110 expand into concentric circles with air therebetween, with the first (inner) portion of the balloon defining the interior channel 115 that is open to the environment outside the balloon, which, in this case, comprises the interior of the lumen of the intestine, the within the channel 108 and extending to the second end 103. When the balloon 110 is deflated and air is evacuated therefrom, the first and second portions of the balloon 110 are drawn radially inward and together to close off the interior channel.

The balloon 110 extends from a first end 111 (e.g., a proximal end) that, when the device 100 is implanted in the patient, extends proximally into the intestine to a second end 112 (e.g., distal end) that is wrapped around the body 104 of the stent 101. Thus, the balloon 110 comprises a first region 113 that extends loosely and proximally from the first end 102 of the body 104 and a second region 114 attached to the body 104 that extends around the exterior of the balloon 110. The second region 114 remains in substantially fixed location relative to the stent 101 in both the inflated state and the deflated states. In the inflated state, the first region 113 defines a tube, as described above. Thus, when inflated, the first region 113 of the balloon 110 and the body 104 of the stent 101 (around which the second region 114 of the balloon 110 is disposed) form a continuous channel through which waste can pass.

When the balloon 110 is deflated, the tube is evacuated which collapses the first region 113 of the balloon 110 (e.g., drawing together the balloon material on opposing sides of the tubular cross section), while the second region 114 remains cylindrical due to the exterior of the body 104 of the stent 101 to which it is attached. The collapsed first region 113 of the balloon 110 forms a seal effectively blocking access from the lumen of the intestine to the channel 108 at the first end 102 of the device 100.

The balloon may be formed of materials such as Silicone which are robust to the cyclic stresses of repeated inflation and deflation. In the event that the balloon requires replacement, the balloon may be detached from the stent and a new one may then be inserted in its place. The balloon may be attached to the stent using, for example, an adhesive, such as biocompatible glue. Although the balloon of FIG. 1 is placed on the outside of the stent, the balloon may alternatively be placed internal to the stent, e.g., for ease of replacement. In another embodiment, the balloon can have a solid proximal segment that is not inflatable but can be part of the balloon to facilitate easier attachment to and detachment from the stent. For example, as would be understood by those skilled in the art, the proximal segment may include an attachment or locking mechanism such as hooks, ball/hole, bayonet, thread, etc., to attach the balloon to the stent.

FIG. 2a shows a side view of the device 100 of FIG. 1 with the balloon 110 deflated to close the channel 108 through the stent 101. When the balloon 110 is deflated, the first region 113 of the balloon 110 extending proximally from the first end 102 of the stent 101 into the lumen is drawn together such that the waste (e.g., the stool) cannot pass into the interior channel 115 of the balloon 110. The balloon 110 effectively acts as a closure mechanism preventing the waste leakage from the device 100.

FIG. 2b shows a side view of the device 100 of FIG. 1 with the balloon 110 inflated and the channel 108 through the stent 101 opened. When the balloon 110 is inflated, the first region 113 of the balloon 110 is expanded into its tubular shape with an open proximal end so that the waste can pass from the lumen of the intestine through the interior channel 115 of the balloon 110 and the channel 108 of the stent 101 so that it can be evacuated out the second end 103.

It is noted that in FIGS. 2a-b, the balloon is internal to the stent while in FIG. 1, the balloon is external to the stent.

The balloon 110 according to an exemplary embodiment has an opening 116 (e.g., any known inflation port such as shown in FIGS. 2a-b) on its second end 112 to which a tubing 120 can be attached for inflating and deflating the balloon 110. In the example of FIGS. 1 and 2a-b, the balloon 110 is shown with the tubing 120 extending directly from the opening 116 to a location external to the device 100. A user of the device 100 can inflate/deflate the balloon 110 manually (e.g., using a syringe) or automatically (e.g., using a pump) to be described in greater detail below.

Returning to FIG. 1, the flared portion 106 of this embodiment defines an enlarged opening at the second end 103 of the stent 101 through which the waste can be evacuated. As would be understood by those skilled in the art, the flared portion 106 may be secured to the stoma and/or skin surrounding the stoma using sutures or any other suitable material or devices. The stent 101 can be secured to the stoma area to keep the lumen patent at the distal location while the stitching and flare of the stent 101 prevent migration of the device 100 distally.

A cap-like component (referred to herein as a “cap” for ease of explanation) (not shown in FIG. 1) can be used to seal off the stent 101 to prevent leakage from the channel 108. As would be understood by those skilled in the art, the cap of this embodiment may be configured in any of a number of known ways to mate with the flared portion 106 from the second end 103 including press-fit, screw connection, snap-fit, clasped, or other manners of attachment. The cap can be attached to the stent 101 by the user during normal use until the user detaches the cap to evacuate waste.

FIG. 1b shows an ostomy device 130 including a cap 135 according to various exemplary embodiments. The ostomy device 130 is similar to the ostomy device 100 described in FIG. 1a and includes a stent 131 and a balloon 133. In this example, the stent 131 includes an edge 132 at the second end 103 (distal end of the flared portion 106) to prevent waste from running or wicking onto the skin, which can cause skin irritation. The edge 132 also aids cleaning of the flared portion 106. The balloon 133 includes a solid proximal portion 134 that facilitates attachment of the balloon 133 to the stent 131. The cap 135 of this example comprises a plug that configured to be inserted within the proximal portion 134 of the balloon 133.

In some embodiments, the cap includes a luer attachment for inflation and deflation of the balloon for evacuation and subsequent closure. The opening 116 of the balloon can be internal to the stent 101, e.g., within the cover 107, and may include tubing extending therefrom that can be coupled, for example, to an opening in the cap. The other side of the opening comprises a luer lock or other connector, to which the user may attach a syringe or other inflation/deflation device.

FIG. 1c shows an ostomy device 140 including a cap 145 with an inflation tube 146 extending proximally from the cap 145 to a balloon 143 according to various exemplary embodiments. The ostomy device 140 is similar to the ostomy device 130 described in FIG. 1b and includes a stent 141 and a balloon 143. The stent 141 includes an edge 142 at the transition between the body and the flared portion, similar to the arrangement shown in FIG. 1b. The balloon 143 includes a solid proximal portion 144 that facilitates attachment of the balloon 143 to the stent 141 and the cap 145 comprises a plug that configured to be inserted within the proximal portion 144 of the balloon 143, similar to the arrangement shown in FIG. 1b. In this example, an inflation tube 146 extends from the balloon 143 and connects to the cap 145 so that the balloon 143 can be deflated via the cap 145. Although the balloon 143 could also be inflated with the cap 145 inserted in the device 140, it may be preferable to inflate the balloon 143 and open the internal channel with the cap off so that stool can pass through the internal channel and out of the proximal end.

The device would also have the necessary components in an accessory kit that the patient would carry around with them (e.g., small syringe, small tubing, wipes, and sterile spray).

In another embodiment, the device may be inflated using a reservoir and an implanted pump like the Boston Scientific AMS 800™ Artificial Urinary Sphincter hence eliminating the need for manual inflation.

In another embodiment, the device may also be placed external to the intestine so that the balloon closes around the circumference of the lumen to close off the vessel. In this case, the balloon may be separated from the stent. In this case, the balloon is placed over the intestine before the intestine is attached to the stoma. The balloon can be a ribbon shape that is wrapped around the intestine and secured to itself.

FIG. 3a shows a side view of an ostomy device 150 including a stent 151 placed external to a lumen 153 of the intestine with the balloon deflated and the channel 152 through the stent 151 closed. When the balloon is deflated, the first region of the balloon extending proximally from the first end of the stent 151 around the lumen is drawn together such that the lumen is closed and the waste cannot pass therethrough.

FIG. 3b shows a side view of the device 150 of FIG. 3a placed external to the lumen 153 with the balloon inflated and the channel 152 through the stent 151 opened. When the balloon is inflated, the first region of the balloon is expanded into its tubular shape so that the lumen 153 remains open and the waste can pass through the lumen 153 and through the channel 152 of the stent 151 for evacuation from the second end of the device 150.

An ostomy device according to another embodiment includes an implant comprising a body and an external flange attached to the body for securing the implant to the stoma (e.g., to an external surface of the skin surrounding the ostomy). The body includes a tubular portion that defines a channel through which the waste can be passed through the stoma. The body further includes a living hinge so that, once implanted as desired, a proximal portion of the body can be expanded into a flared shape in which the proximal end contacts the wall of the intestine adjacent to the stoma lumen. A stoma plug can then be inserted into the channel of the body to prevent the flow of the waste and can be removed to expel the waste in the intestine.

FIGS. 4-8 show views of the relevant anatomy 250 of a patient at a stoma site and the deployment of an ostomy device 200 therein. The device 200 comprises an implant 201 including a body 202 configured for insertion within a stoma and an external flange 210 for attaching the device 200 to the skin surrounding the stoma to fix the position of the implant 201 in the stoma. The device 200 further includes a rubber ring 220 at the attachment surface between the flange 210 and the skin and a stoma plug 230 for sealing the channel 205 of the implant 201. It should be understood that FIGS. 4-8 show sectional views and that various aspects of the anatomy 250 and the device 200 may be tubular or conical, e.g., have circular cross-sections in planes transverse to their longitudinal axes as will be described in more detail below. However, those skilled in the art will understand that the geometry of the channels of these devices may be in any desired shape so long as the dimensions are sufficient to permit the passage therethrough of the waste that needs to be evacuated.

FIG. 4 shows the anatomy 250 of a patient including the intestines 251, a stoma 253, and skin 260 of the abdominal wall. The stoma 253 defines a stoma channel 254 extending from a region 252 of the wall of the intestines 251 internal to and adjacent to the stoma channel 254 to a stoma opening 255. The stoma 253 protrudes from the skin 260 and includes a portion 256 external to the skin 260 that is stitched to the skin 260. It should be understood that FIG. 4 shows a sectional view and that the stoma 253, the stoma channel 254, the region 252, the stoma opening 255 and the portion 256 of the stoma 253 are generally tubular or conical. A physician can insert the device 200, specifically, a body 202 of an implant 201 to be described below, via the stoma opening 255.

FIG. 5 shows an implant 201 of the device 200 including a body 202 defining a channel 205 and the flange 210 configured for attaching the implant 201 to the anatomy 250. The body 202 is sized and shaped for implantation within the stoma 253 via the stoma channel 254. In particular, the body 202 extends from a first end 203 that, when the device 200 is implanted in the patient as desired, is located within the intestines 251 to a second end 204 that, when the device 200 is implanted as desired, is located exterior to the stoma opening 255.

The body 202 includes a living hinge 207 configured transition between a first state (e.g., an insertion state) and a second state (e.g., a deployment state) that will be described below with regard to FIG. 6. A first portion 206 of the body 202 (e.g., a proximal portion) extends from the first end 203 to the living hinge 207 while a second portion 208 of the body 202 (e.g., a distal portion) extends from the living hinge 207 to the second end 204. The living hinge 207 is biased so that, when no external forces are imposed on the body 202, the hinge 207 bends and opens radially so that the first portion 206 of the body 202 flares radially outward into a shape that, in this embodiment, corresponds to a frustum of a cone.

In the insertion state, the living hinge 207 is restricted from expansion by a sheath (not shown) disposed around the body 202. Thus, in the insertion state, the body 202 is substantially tubular to facilitate the insertion through the stoma channel 254. The body 202 can be formed of various materials including, e.g., plastic or titanium. The body 202 can also be formed of nitinol (e.g., a nitinol stent structure configured to permit the serosa of the intestine to grow therein).

The implant 201 of this embodiment further comprises the flange 210 comprising a collar portion 211 having a substantially conical shape (e.g., the frustum of a cone) corresponding to the substantially conical shape of the portion 256 of the stoma 253. On a first end of the collar portion 211 (e.g., a distal end) that has a diameter smaller than that of the proximal end of the collar portion 211, is a first rim 212 that is fixed to the second portion 208 of the body 202 at a location that remains external to the stoma 253 when the body 202 is inserted through the stoma 253.

As would be understood by those skilled in the art, the first rim 212 may be attached to the body 202 (e.g., using at least one screw or glue or any other known method). On a second end of the collar portion 211 (e.g., a proximal end thereof) having a diameter larger than that of the first end of the collar portion 211, is a second rim 213 that can be sutured to the skin 260 at a skin location 261 adjacent to and surrounding the portion 256 where the stoma 253 is stitched to the skin. The flange 210 of this embodiment may be formed of any suitable materials (e.g., including titanium).

As described above, in use, a physician may insert the body 202 of the implant 201 into a previously created stoma 253 with the implant 201 in the insertion state, e.g., with a sheath over the body 202 and the living hinge 207 restricted from expansion. The attached flange 210 follows the body 202 until the first rim 212 is brought into abutting contact with the portion 256 of the stoma 253 exterior to the skin 260, whereupon the second rim 213 can be attached to the skin location 261 adjacent to the stoma 253, e.g., via sutures. A rubber ring 220 may optionally be placed proximal to the second rim 213 against the skin location 261 of the skin 260 to provide a seal to more effectively prevent leakage and to reduce patient discomfort at the skin, as shown in FIG. 6 below.

Due to the rubber ring 220, there will be less surface area of contact between the second rim 213 and the skin 260 to reduce the skin discomfort relative to, e.g., adhesives or other methods of attachment. After inserting and attaching the implant 201, the operating physician may remove the sheath around the body 202 so that the living hinge 207 can open. It should be understood that FIGS. 5-6 show sectional views and that the body 202, the collar portion 211 and the rubber ring 220 of these embodiments are generally tubular, conical, or circular but that other shapes (e.g., elliptical or oval cross-sectional shapes) may also be employed.

FIG. 6 shows the device 200 including the implant 201 of FIG. 5 with the body 202 in the deployment state, e.g., with the living hinge 207 bent so that the first portion 206 (e.g., proximal portion) of the body 202 can expand into a flared shape. In the deployment state, the first portion 206 is expanded into a frustum of a cone to create a surface for contacting the region 252 of the intestines 251 internal and adjacent to the stoma 253. This surface allows the implant 201 to stay inside the intestines 251 and prevents movement of the implant 201 due to peristalsis movement.

Accordingly, the deployed implant 201 is fixed to the location of the stoma 253 and can provide a proper channel and connection for stoma collection. Due to the positive locking between the body 202 and the flange 210 there will be less chance of leakage. Due to the force between these parts in the deployed state, the rubber ring 220 provides enhanced sealing around the stoma area.

The device 200 further includes a stoma plug 230, as shown in FIGS. 6-8. After deployment of the implant 201, the stoma plug 230 may be inserted into the second end 204 of the body 202. The stoma plug 230 can be attached inside the body 202, e.g., in the channel 205, via features such as a press fit, a screw mechanism, a snap fit or any other known suitable mechanism. In some embodiments, the stoma plug 230 may be disposable.

FIG. 7 shows the stoma plug 230 of the device 200 of FIG. 6. The stoma plug 230 includes a body 231 and a membrane 232 for the release of gas built up in the intestines. The membrane 232 can comprise a sheet (e.g., formed of PTFE or any other suitable biocompatible material) with slits extending therethrough to enable gas to escape. This may lower gas pressure thereby aiding in the prevention of leakage of fecal content due to gas buildup in the intestines. During normal everyday usage, the device 200 may include the inserted stoma plug 230. As would be understood by those skilled in the art, when the patient intends to expel the waste, the stoma plug 230 can be removed and the channel 205 through the body can be opened.

FIG. 8 shows the removal of the stoma plug 230 of FIG. 6 for the passing of the waste. When the patient intends to pass the waste they can attach a stoma bag 240 to the second end 204 of the implant 201. In this example, the stoma plug 230 is press fit into the channel 205. To remove the stoma plug 230, the patient can apply pressure around the body 202 at a location along its length proximal to the stoma plug 230, e.g., by pressing down on the flange 210 as shown in FIG. 8, so that the body 202 deforms slightly to apply a distal force on the stoma plug 230 and push it out of the channel 205 through the second end 204 of the body 202. The stoma plug 230 will fall into the stoma bag 240, opening the channel 205 to allow the waste to passed into the stoma bag 240. After the waste is collected, the patient can remove the stoma bag 240 and insert a new stoma plug 230.

In other embodiments, the stoma plug 230 may be attached in other ways to the implant 201 (e.g., screwed, snap fit, clasped, etc.). In some embodiments, the stoma plug 230 can be reusable. In some cases, an ostomy bag is not used and the waste can be passed into another type of waste receptacle as would be understood by those skilled in the art.

An ostomy device according to another embodiment includes an implant that creates an internal connection directly from the intestine and through a hole in the abdomen of the patient to the environment exterior to the patient. In these embodiments, a stoma is not created and the implant forms the only channel from the intestine for passing waste. A waste receptacle, e.g., ostomy bag, can be attached to the implant. In some embodiments, the implant comprises a stent.

FIG. 9 shows a stent 301 of an ostomy device 300 defining a channel 304 from a first end 302 (e.g., proximal end) configured for connection to the intestines to a second end 303 (e.g., distal end) configured for connection to the environment external to the abdomen. The stent 301 is formed of a wire mesh and comprises a cover. FIG. 10 shows the device 300 including the stent 301 connected to a patient anatomy 350. The patient anatomy 350 includes the intestines 351, an end 352 of the intestines 351 and an abdominal wall 353. The device 300 is implanted in a hole in the abdominal wall 353 of the patient.

The stent 301 comprises a first portion 305 (e.g., proximal portion) extending from the first end 302 to a first flange 306. The first portion 305 is configured for connection to the end 352 of the intestine 351 (e.g., by suturing). The first flange 306 defines a raised portion of the stent 301, e.g., having a greater diameter than the surrounding portions, that is interior to the abdominal wall 353 and can be in abutting contact with an inner surface 354 of the abdominal wall 353. A second portion 307 extends from the first flange 306 to a second flange 308. The second portion 307 of this embodiment extends through the hole in the abdominal wall 353. The second flange 308 defines a second raised portion of the stent that is exterior to the abdominal wall 353 and which is configured to be placed in abutting contact with an outer surface 355 of the abdominal wall 353 in a manner similar to that described above. A third portion 309 (e.g., distal portion) extends from the second flange 308 to the second end 303.

The device 300 further includes a coupling mechanism (not shown) for attaching a waste receptacle, e.g., an ostomy bag 310. The ostomy bag 310 for use with the device 300 would have a corresponding feature for coupling with the coupling mechanism of the device 300. Waste from the intestine 351 can flow directly through the stent 301 to the ostomy bag 310. The stent 301 is covered to prevent leaks.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims.

1-15. (canceled)

16. An ostomy device, comprising:
an implant including a body sized and shaped for implantation in a stoma, the body including a first portion defining a channel extending from a first end to a second end, the first end configured to interface with a lumen interior to an abdomen, the second end configured to provide a path from the lumen through the channel to an exterior of the abdomen,
wherein the implant further includes a flange attached to an exterior of the body, the flange including a collar extending from a first rim attached to an exterior of the body to a second rim attachable to a skin adjacent to the stoma.

17. The device of claim 16, wherein the body comprises a living hinge between the first portion and a second portion and wherein the living hinge is biased to bend so that the second portion assumes a flared shape.

18. The device of claim 17, further comprising:
a sheath around the body configured to restrict the living hinge from bending during insertion of the implant.

19. The device of claim 18, wherein the sheath is removed after the implant is inserted so that the living hinge bends and the second portion flares to contact intestinal walls adjacent to the lumen.

20. The device of claim 16, further comprising:
a rubber ring disposed between the second rim and the skin to provide a lining for leakage prevention.

21. The device of claim 16, further comprising:
a stoma plug configured to insert in the channel at the second end of the body.

22. The device of claim 21, wherein the stoma plug comprises a plug body and a membrane, the membrane including slits to enable gas to pass from the lumen through the channel.

23. The device of claim 21, wherein the stoma plug is press fit into the channel and removable by proving a radial force on the body to deform the body and push the stoma plug out of the channel.

24. The device of claim 23, wherein an ostomy bag is attached around the second end of the body and the stoma plug is pushed into the ostomy bag.

25. The device of claim 21, wherein the stoma plug is screwed, snap fit or clasped to the body.

26. The device of claim 16, wherein the first rim is screwed or glued to the body and the second rim is sutured to the skin adjacent to the stoma.

27. An ostomy device, comprising:
a stent sized and shaped for implantation in a hole in an abdomen, the stent comprising a tubular body defining a channel extending from a first end to a second end, the first end configured to interface with a lumen interior to the abdomen, the second end configured to provide a path from the lumen through the channel to an exterior of the abdomen,
wherein the stent is configured to be attached to the hole in the abdomen.

28. The device of claim 27, wherein the stent comprises a first flange configured to contact an inner surface of the abdomen and a second flange configured to contact an outer surface of the abdomen.

29. The device of claim 27, wherein the first end of the stent is stitched to the lumen.

30. The device of claim 27, wherein the second end includes a coupling mechanism configured to be attached to an ostomy bag.

31. A method, comprising:
implanting an implant of an ostomy device in a stoma, the implant comprising a body including a first portion defining a channel extending from a first end to a second end, the first end configured to interface with a lumen interior to an abdomen, the second end configured to provide a path from the lumen through the channel to an exterior of the abdomen, wherein the implant further includes a flange attached to an exterior of the body, the flange including a collar extending from a first rim attached to an exterior of the body to a second rim; and
attaching the second rim to a skin adjacent to the stoma.

32. The method of claim 31, wherein the body comprises a living hinge between the first portion and a second portion and wherein the living hinge is biased to bend so that the second portion assumes a flared shape.

33. The method of claim 32, wherein the ostomy device further includes a sheath around the body configured to restrict the living hinge from bending during insertion of the implant.

34. The method of claim 33, further comprising:
removing the sheath after the implant is inserted so that the living hinge bends and the second portion flares to contact intestinal walls adjacent to the lumen.

35. The method of claim 31, wherein the ostomy device further includes a rubber ring disposed between the second rim and the skin to provide a lining for leakage prevention.
